(19) United States
(12) Patent Application Publication
(10) Pub. No.: US 2024/0316130 A1
(43) Pub. Date: Sep. 26, 2024

(54) COMPOSITION AND METHODS INCLUDING RECOMBINANT VIRUSES MODULATING ARGININE BIOSYNTHESIS

(71) Applicant: UNM Rainforest Innovations, Albuquerque, NM (US)

(72) Inventor: Eric Bartee, Albuquerque, NM (US)

(21) Appl. No.: 18/609,666

(22) Filed: Mar. 19, 2024

Related U.S. Application Data
(60) Provisional application No. 63/454,547, filed on Mar. 24, 2023.

(51) Int. Cl. A61K 35/768; C07K 14/54; C07K 14/705; C12N 9/00; C12N 9/10; C12N 9/88; C12N 15/86

(52) U.S. Cl. CPC A61K 35/768; C07K 14/5434; C07K 14/70521; C12N 9/1018; C12N 9/88; C12N 9/93; C12N 15/86; C12Y 201/03003; C12Y 403/02001; C12Y 603/04005; C12N 2710/24032; C12N 2710/24043

(57) ABSTRACT

A replication-competent recombinant virus includes an expression cassette encoding a protein associated with arginine biosynthesis. The protein associated with arginine biosynthesis may include argininosuccinate synthase 1 (ASS1), argininosuccinate lyase (ASL), or ornithine transcarboxylase (OTC). The recombinant virus may be derived from a myxoma virus. A method of treating a cell includes contacting a cell with a recombinant virus including an expression cassette encoding a protein associated with arginine biosynthesis. The protein associated with arginine biosynthesis may be ASS1, ASL, OTC.

### CROSS REFERENCE TO RELATED APPLICATIONS

This application claims the benefit of 63/454,547, filed Mar. 24, 2023, the disclosure of which is incorporated by reference herein in its entirety.

### SEQUENCE LISTING

This application contains a Sequence Listing electronically submitted via Patent Center to the United States Patent and Trademark Office as an .xml file entitled “0310.000179US01.xml” having a size of 1.9 kilobytes and created on Mar. 19, 2024. The information contained in the Sequence Listing is incorporated by reference herein.

### SUMMARY

In one aspect, this disclosure describes a recombinant virus, the recombinant virus including an expression cassette encoding a protein associated with arginine biosynthesis, wherein the recombinant virus is replication competent. In one or more embodiments the recombinant virus is derived from an oncolytic virus.

In one or more embodiments, the protein associated with arginine biosynthesis comprises argininosuccinate synthase 1 (ASS1), argininosuccinate lyase (ASL), or ornithine transcarboxylase (OTC).

In one or more embodiments, the virus is derived from a myxoma virus, a herpesvirus (e.g., talimogene laherparepvec), an orthopox virus (e.g., vaccinia virus), an adenovirus, a parvovirus, an alphavirus (e.g., Semliki Forest virus), a flavivirus (e.g., zika virus), a paramyxovirus (e.g., measles virus, Newcastle disease virus), a picornavirus (e.g., Seneca Valley virus), a poliovirus, a reovirus, a rhabdovirus (e.g., vesicular stomatitis virus), or a filovirus (e.g., Marburg virus).

In one or more embodiments, the recombinant virus includes at least two expression cassettes that cumulatively, encode a protein associated with arginine biosynthesis, a soluble form of programmed cell death protein 1 (PD1), and interleukin 12 (IL-12). In one or more of these embodiments, two of the expression cassettes are provided as a dicistronic expression cassette.

In another aspect, this disclosure describes a composition that generally includes a first recombinant virus that includes an expression cassette encoding a protein associated with arginine biosynthesis and a second recombinant virus that includes an expression cassette encoding PD1 and IL-12.

In one or more embodiments, the protein associated with arginine biosynthesis comprises argininosuccinate synthase 1 (ASS1), argininosuccinate lyase (ASL), or ornithine transcarboxylase (OTC).

In one or more embodiments, at least one recombinant virus in the composition is derived from a myxoma virus.

In another aspect, this disclosure describes a method of treating a cell. Generally, the method includes contacting a cell with a recombinant virus that includes an expression cassette encoding a protein associated with arginine biosynthesis.

In another aspect, this disclosure describes a method of increasing replication of an oncolytic virus in a tumor microenvironment. Generally, the method includes introducing to the tumor microenvironment a recombinant virus that includes an expression cassette encoding a protein associated with arginine biosynthesis.

In one or more embodiments, the protein associated with arginine biosynthesis is argininosuccinate synthase 1 (ASS1), argininosuccinate lyase (ASL), or ornithine transcarboxylase (OTC).

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE DRAWINGS

FIG. 1. Myxoma virus treatment alters arginine metabolism. B16F10 tumors established in C57Bl/6J mice were treated with either PBS or MyxGFP. Four days post treatment, tumors were harvested and immediately snap frozen in liquid nitrogen. Whole tumor homogenates were then used to quantify a set of 226-metabolite species via LC-MS. n=5-7 tumors per group. (A) Metabolite Set Enrichment Analysis of LC-MS results. (B) Viral yields from B16F10 cells grown in DMEM lacking various single amino acids for 24 hours and infected with MyxGFP at an MOI=5. Cells were harvested 24 hours post-infection for viral quantification. (C) Heatmap of metabolite concentrations involved in the urea cycle and polyamine biosynthesis from experiment (A). Color indicates -Log₁₀(Holm adj. p value). Arrows indicate significant (Holm adj. p value <0.05) increases or decreases in MyxGFP treated cohort relative to Mock cohort. Gray indicates species not evaluated in this panel.

FIG. 2. Bioavailable Arg facilitates Myxoma virus replication. (A) The indicated cells lines were incubated in complete media or media lacking Arg for 24 hours and then infected with MyxGFP at an MOI=5. Expression of virally derived GFP was visualized 24 hours post-infection using fluorescent microscopy. (B) The indicated cells were incubated in complete media or media lacking Arg for 24 hours and then infected with MyxGFP at an MOI=5. At the indicated times post-infection, cells were harvested and the amount of infectious virus present quantified using viral titration assays. Statistical significance was determined by unpaired Student's T-test on samples collected at 24 hours (*** p<0.001). n=3 per group, per cell line. (C) The indicated cells were incubated in a gradient of Arg-deficient DMEM for 24 hours prior to infection with MyxGFP at an MOI=5. Cells were harvested 24 hours post infection and the amount of infectious virus present quantified using viral titration assays. n=3 per group, per cell line.

FIG. 3. B16/F10 tumors contain an amount of arginine that can restrict myxoma virus replication. (A) Quantitation of the amount of infectious viral progeny from B16/F10 cells infected with vGFP for 24 hours in the presence of various amounts of arginine. Vertical lines indicate the previously reported abundance of arginine in mouse serum (50-150 μM). (B) Mice were implanted with B16/F10 tumors, which were allowed to establish for seven days. The indicated tissues were then harvested and the abundance of arginine was quantitated using ELISA.

FIG. 4. Generation of functionally ASS1^{-/-} B16/F10 cells. ASS1 was functionally ablated from B16/F10 cells using CRISPR/Cas9 genome editing resulting in two functionally ASS1^{-/-} clones. (A) Overall cell morphology of ASS1^{WT} and ASS1^{-/-}-cells in the presence of arginine. (B) Western blot analysis of ASS1 protein from each clone. Note that the functionally ASS1^{-/-} clones still express ASS1 protein but the size of this protein is reduced compared to WT. (C) Growth of WT and ASS1^{-/-} cell in media containing 400 μM arginine (media), media with no arginine, or media with no arginine supplemented with either citrulline or argininosuccinate. Cell growth data is normalized to cell counts in media controls.

FIG. 5. Intrinsic arginine biosynthesis can support myxoma virus replication in Arg limited conditions: (A) Western blots analyzing expression of the urea cycle components ASS1, ASL, and OTC in B16F10 and A9F1 cells. (B) MTT assays of B16F10 and A9F1 cells cultured for 24 hours in control DMEM (Control), Arg-deficient media supplemented with 400 μM citrulline (−Arg/+Cit), or Arg-deficient media (−Arg). (C) The indicated cells were incubated as above and then infected with MyxGFP at an MOI=5. 24 hours post-infection the amount of infectious virus present was quantified using viral titration assays. Statistical significance in (B) and (C) determined by ANOVA with Tukey's HSD post-hoc test (*** p<0.001). n=3 per group, per cell line.

FIG. 6. Loss of ASS1 reduces MYXV replication in vivo. ASS1^{WT} or ASS1^{KO} (KO #1 and KO #4) B16F10 tumors were established on C57Bl/6J mice and then treated with a single bolus of 1×10⁶ FFU of MyxGFP. Six days post infection, tumors were harvested and processed for frozen sectioning and quantification of viral titer. (A) Images of tumor sections that displayed the median amount of infection within each cohort. GFP signal indicates area of infection. (B) Quantification of infection area in infected tissues seen in (A) displayed as a percent of total tumor area which is GFP. One section was taken for imaging per tumor/mouse. n=6-9 per group. Hashed bars indicate mean value for each group. (C) Quantification of infectious virus present within tumors. n=6-9 per group. Hashed bars indicate mean value for each group. Statistical significance in B and C was determined by ANOVA with Games-Howell post-hoc test (* p<0.05, ** p<0.01, *** p<0.001).

FIG. 7. Myxoma virus-based oncolytic virotherapy is less effective in ASS1^{-/-} tumors. ASS1^{WT} or ASS1^{KO} (KO #1) tumors were established in C57Bl/6J mice and then treated with three doses of either PBS (mock) or 1×10⁵ FFU of MyxPD1/IL 12 intratumorally QOD over 5 days. Tumor burden was then monitored every other day in all cohorts. (A) Schematic of treatment regimen. (B) Spaghetti plots tracking individual tumor sizes over time. (C) Overall survival of mice. Statistical significance was determined using Log-Rank Analysis (*=p<0.05, ns=no significance)

FIG. 8. Generation of an ASS1-expressing myxoma virus. (A) Genomic Schematic of recombinant viruses used in this study including MyxASS1 and MyxASS1^{FS}. (B) Expression of murine ASS1 in BSC40 cells infected with either MyxASS1 or MyxASS1^{FS} for 24 hours.

FIG. 9. Expression of exogenous ASS1 from a recombinant MYXV improves viral replication in ASS1^{-/-} tumors. (A) The indicated cells were incubated in complete media, −Arg media, or −Arg media supplemented with citrulline for 24 hours and then infected with either MyxASS1^{FS} (left) or MyxASS1 (right) at an MOI=5. 24 hours post infection, cells were harvested and the amount of infectious virus present quantified using viral titration assays. Statistical significance determined by unpaired Student's T-test (*** p<0.001, n.s.=no significance). n=3 per group, per cell line. (B) ASS1^{WT}, ASS1^{KO} (KO #1 and KO #4) tumors were established in C57Bl/6J mice. Tumors were then treated with a single bolus of 1×10⁶ FFU of either MyxASS1 or MyxASS1^{FS}. Six days post infection, tumors were harvested, and the abundance of infectious virus quantified using viral titer assay. n=3-6 tumors per group. Statistical significance determined by unpaired Student's T-test (specific p values given). (C) A9F1 tumors were established in C57Bl/6J mice and treated as in D. Statistical significance determined by unpaired Student's T-test (*** p<0.001). n=5-6 tumors per group.

FIG. 10. Genomic Schematic of recombinant viruses used in this study including MyxPD1/IL12 and MyxASS1/PD1/IL12.

FIG. 11. Expression of ASS1 improves MYXV-based OV: ASS1^{WT}, ASS1^{KO} (KO #1 and KO #4), and A9F1 tumors were established in C57Bl/6J mice and then treated with three doses of either PBS (mock) or 1×10⁵ FFU of MyxPD1/IL12 or MyxASS1/PD1/IL12 intratumorally QOD over 5 days. Tumor burden was then monitored every other day in all cohorts. (A) Spaghetti plots tracking individual tumor sizes over time. (B) Overall survival of mice. Statistical significance was determined using Log-Rank Analysis (*=p<0.05). n=6-9 mice per group.

### DETAILED DESCRIPTION OF ILLUSTRATIVE EMBODIMENTS

This disclosure describes tools and methods for use in oncolytic virotherapy. More particularly, this disclosure describes cell lines and viruses for use studying the impact of cellular metabolites, such as arginine, on the efficacy of oncolytic virotherapy.

Oncolytic virotherapy is used to treat a variety of solid tumors. While much of the anti-tumor activity of oncolytic virotherapy is ultimately mediated by the induction of T cell responses, the success of oncolytic virotherapy relies on the in vivo replication of the viral agents within infected tumor cells. Understanding the fundamental mechanisms that govern the intratumoral replication of oncolytic agents is therefore beneficial to improving clinical application of these therapies.

Viruses, including oncolytic viruses, rely on host metabolites and resources for their propagation. Metabolic dysregulation is a known hallmark of cancer, and some metabolic changes are relatively consistent across different types of cancers. For example, a wide variety of cancers, including melanoma, lung cancer, ovarian cancer, hepatocellular carcinoma, renal carcinoma, osteosarcoma, prostate cancer, and pancreatic cancer, have been shown to epigenetically silence expression of the urea cycle enzyme argininosuccinate synthase 1 (ASS1). Loss of ASS1 prevents cells from synthesizing their own arginine making them entirely reliant on exogenous supplies of this amino acid to maintain their cellular amino acid pools. Since most solid tumors are thought to have limited supplies of exogenous arginine, this creates a tumor microenvironment in which the bioavailability of arginine is extremely limited.

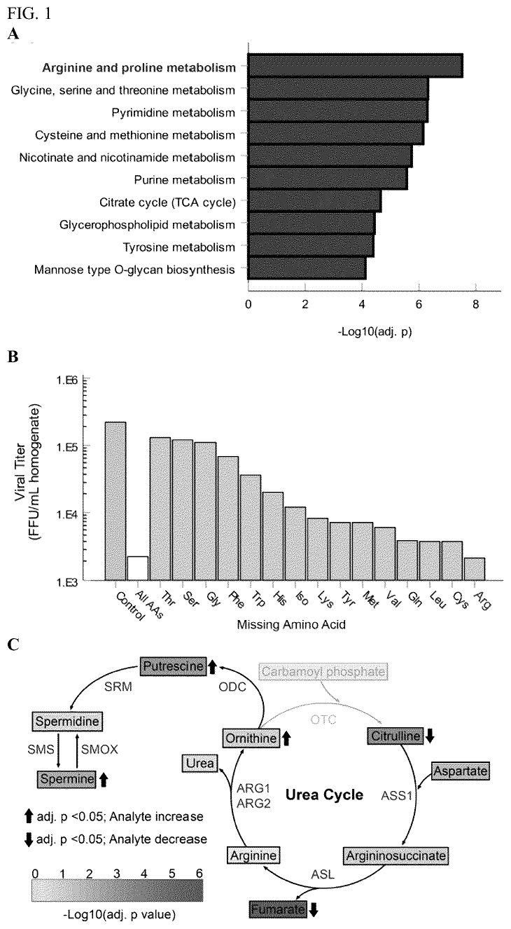

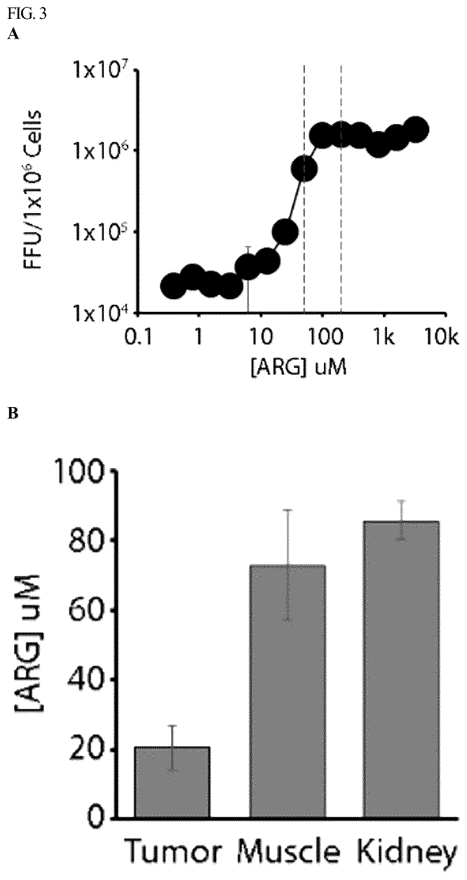

In the absence of bioavailable exogenous arginine, most cells can synthesize arginine de novo as part of the urea cycle (FIG. 1C). Outside of the liver, which is the only tissue to express high levels of ornithine transcarboxylase, the rate-limiting step in this process is the generation of the direct arginine precursor argininosuccinate from citrulline and aspartate by argininosuccinate synthetase 1 (ASS1). As is described herein, solid tumors often have limited supplies of exogenous arginine (FIG. 3B). Thus, tumors often lack or are severely limited in the amount of available exogenous arginine and the ability to synthesize arginine. This results in generally low levels of arginine in and around tumors.

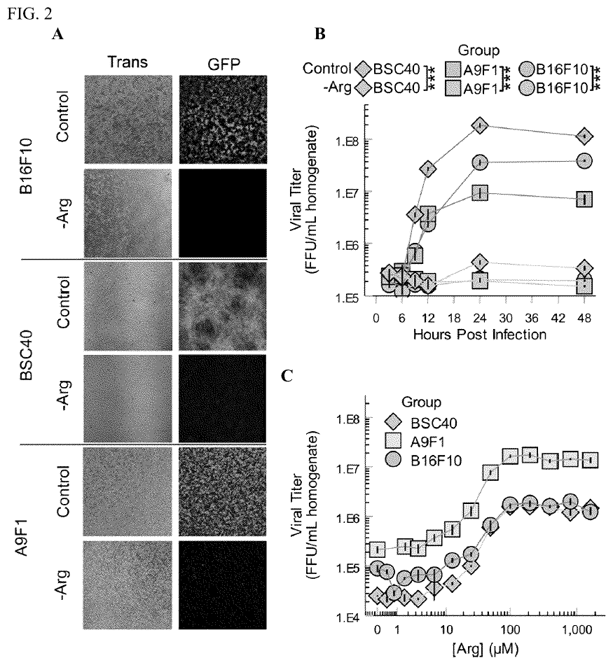

This disclosure demonstrates that MYXV replication is dependent on bioavailable arginine. Oncolytic virotherapy frequently relies on the replication of the oncolytic agent within treated tumors. Additionally, viral replication depends on the metabolic state of their host cells. However, relatively few studies have examined how intratumoral metabolism (particularly outside the context of energetics) might affect oncolytic virotherapy. To identify metabolic pathways that involved in replication of oncolytic MYXV, changes to intratumoral metabolites were assayed following viral treatment. Briefly, B16F10 tumors were established on syngeneic mice and then treated with a single bolus of PBS or 1×10⁶ FFU of MyxGFP. Four days post-treatment, tumors were harvested and the abundance of various hydrophilic metabolites analyzed using whole tissue metabolomics. Metabolite set enrichment analysis of the resulting dataset revealed that viral treatment altered numerous metabolic pathways involved in metabolism of amino acids including arginine, proline, glycine, serine, threonine, cysteine, and methionine, suggesting that these pathways are involved in MYXV infection (FIG. 1A). Consistent with this hypothesis, removal of amino acids from cellular growth media significantly reduced the numbers of infectious MYXV progeny produced in B16F10 cells in vitro (FIG. 1B). Notably, of all the amino acids tested, removal of arginine had the most potent inhibitory effect on viral yields in vitro and arginine metabolism was the most highly altered metabolic pathway identified by the in vivo metabolic screen, with virally treated tumors displaying reduced abundance of the urea cycle precursor L-citrulline as well as increased abundance of the downstream products ornithine, putrescine, and spermine (FIG. 1C). Consistent with arginine's status in MYXV replication, removal of exogenous arginine from cell culture media resulted in a dramatic reduction in the abundance of virally-derived GFP (FIG. 2A) and little-to-no production of new infectious viral particles at any time post infection (FIG. 2B) in multiple cell lines including malignant B16F10 cells, malignant A9F1 cells, and non-malignant BSC40 cells. These changes could not be explained by a general loss of cellular viability since arginine deprivation resulted in cytostasis but did not appear to acutely kill cells over the duration of these experiments. Titrations of exogenous arginine into culture media revealed a strong dose dependency between the production of new infectious MYXV progeny and the bioavailability of this amino acid in all three cell lines with maximal viral replication occurring at concentrations of at least 100 μM and a consistent EC₅₀ of ˜50 μM (FIG. 2C, FIG. 3A). This EC₅₀ is within the physiological range of arginine, which is between 50-150 μM in the serum of healthy mice. Taken together, these data provide evidence of the status of arginine metabolism in the in vitro and in vivo replication of oncolytic MYXV.

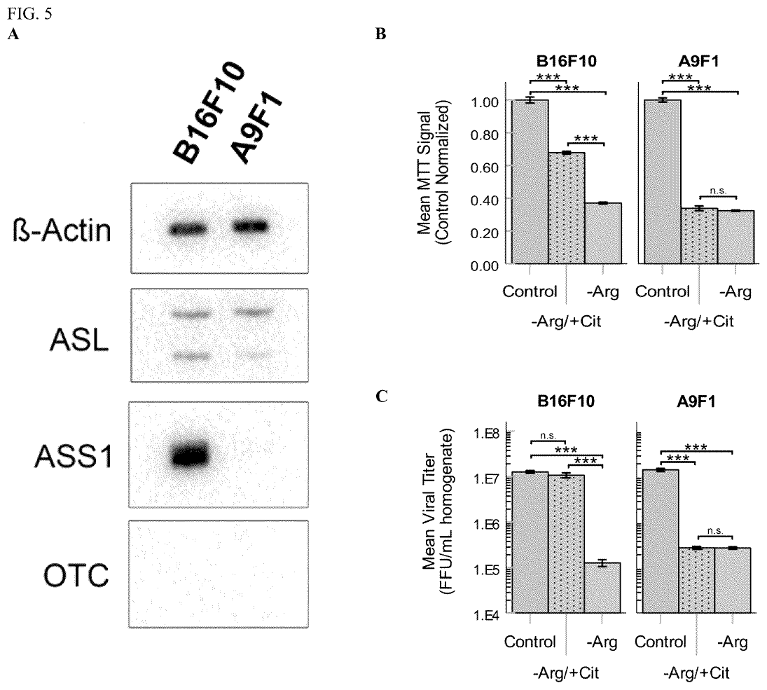

Further, intrinsic arginine biosynthesis can support MYXV replication in ASS1-competent cells. In the absence of exogenous arginine, many cells can directly biosynthesize this amino acid through a portion of the urea cycle. This biosynthesis requires the primary metabolic precursors citrulline and aspartate as well as expression of the urea cycle enzymes ASS1 and ASL (FIG. 1C). Since bioavailable arginine facilitates MYXV replication, the ability of intrinsic arginine biosynthesis to rescue viral replication in the absence of exogenous arginine was tested. First, expression of urea cycle enzymes in B16F10 cells and A9F1 cells were tested using western blotting. The results showed that the murine-lineage B16F10 cells evaluated here expressed both ASS1 and ASL (FIG. 5A). In contrast, A9F1s were found to be deficient in ASS1 expression. Neither cell line expressed detectable levels of ornithine transcarbamoylase (OTC), which is typically only found in the liver. In agreement with these phenotypic results, the cytostasis induced in B16F10s by removal of arginine from the culture medium could be partially rescued by supplementation with exogenous citrulline (FIG. 5B), suggesting that these cells were functionally Arg-autotrophic. In contrast, cytostasis of the intrinsically ASS1-A9F1 cells could not be rescued by citrulline (FIG. 5B). Citrulline supplementation could also rescue MYXV replication in B16F10 cells but not A9F1 cells (FIG. 5C), suggesting that intrinsic arginine biosynthesis is sufficient to support MYXV replication in the absence of exogenous arginine, but that this rescue involves expression of ASS1.

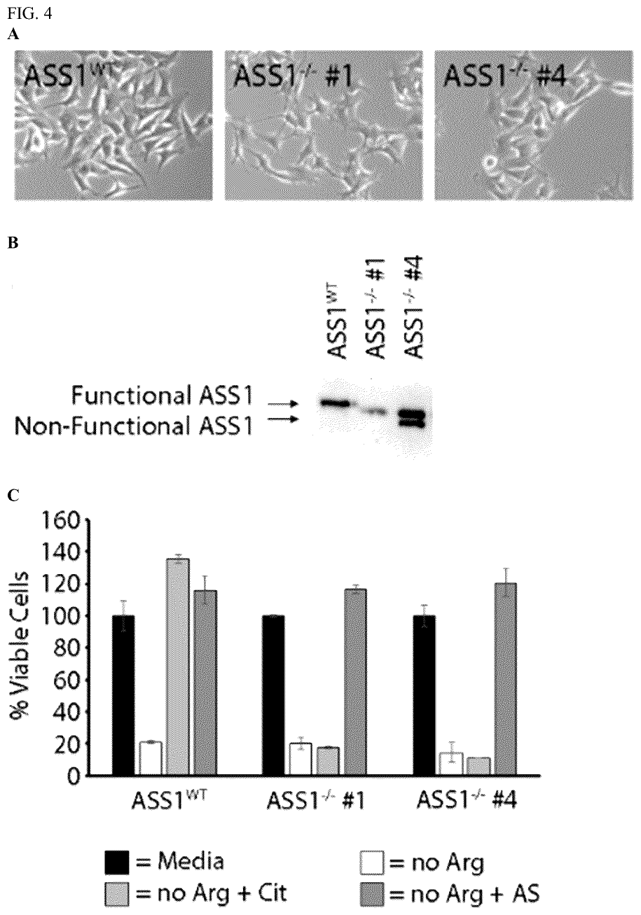

Loss of ASS1 decreases MYXV replication both in vitro and in vivo. Arginine bioavailability can be limiting within many forms of solid tumors so that biosynthesis may be involved in determining the outcomes of MYXV replication under these conditions. Thus, the extent to which a tumor's ASS1-status might influence its responsiveness to MYXV-based oncolytic virotherapy was tested. A series of functionally ASS1-deficient B16F10 cell lines were generated using CRISPR/Cas9 genome editing (FIG. 4A-C). These cell lines exhibited normal growth and morphology when cultured in standard DMEM but produced a truncated version of the ASS1 protein. Both the cellular cytostasis and reduced MYXV replication induced by removal of arginine could no longer be rescued by adding citrulline in these cell lines but remained rescuable by adding the downstream metabolite argininosuccinate (AS), demonstrating that these cells were now functionally ASS1-deficient. Additionally, tumors formed from these ASS1^{KO} cells displayed metabolomic profiles consistent with defective arginine biosynthesis including significantly increased citrulline content and reduced levels of arginine.

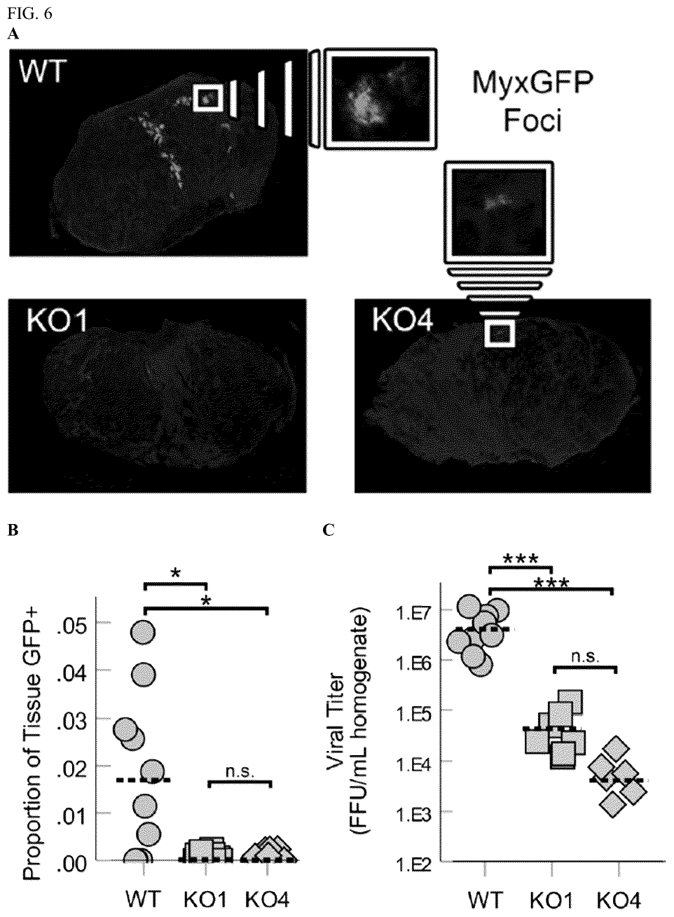

Having generated functionally ASS1-deficient cell lines, the influence of the loss of ASS1 on MYXV replication in vivo was tested. Syngeneic mice were implanted subcutaneously (SQ) with either ASS1^{WT} or ASS1^{KO} B16F10 cells. Seven days post implantation, the resulting tumors were treated with a single bolus of 10⁶ FFU of MYXV. Six days after viral treatment, tumors were harvested and the rate of viral infection was determined both by visually assessing expression of virally derived GFP within tumor sections as well as by quantifying the abundance of infectious virions. Six days post-treatment, ASS1^{WT} B16F10 tumors displayed numerous distinct GFP regions corresponding to individual foci of infection (FIG. 6A, FIG. 6B) and contained high numbers of infectious MYXV particles (FIG. 6C). In contrast, tumors formed from two distinct ASS1^{KO} clones displayed significantly reduced visual signs of infection (FIG. 6A, FIG. 6B) as well as an approximately two-log reduction in infectious virus (FIG. 6C). Taken together, these results suggest that the loss of ASS1 from malignant cells negatively affects the replication of oncolytic MYXV in vivo by preventing intrinsic arginine biosynthesis.

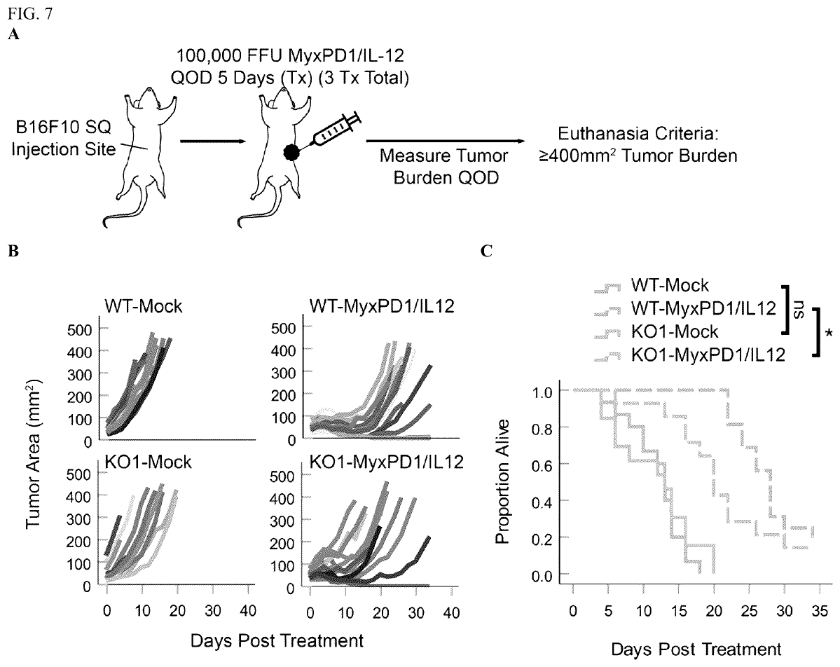

Loss of ASS1 decreases the therapeutic response to MYXV-based oncolytic virotherapy. In MYXV-based oncolytic virotherapy, the oncolytic MYXV replicates within a tumor to induce therapeutic regression. Since loss of ASS1 severely compromised MYXV replication within treated tumors, the extent to which ASS1″ tumors display reduced responsiveness to MYXV-based oncolytic virotherapy was evaluated. Since the therapeutic response of B16F10 tumors to unmodified MYXV is typically negligible, B16F10 tumors were established in syngeneic mice and then the mice were treated with a doubly recombinant MYXV construct that expressed both a PD1 inhibitor and IL 12 (MyxPD1/IL12 (Bartee et al., Cancer Research, 2017, 77(11):2952; Valenzuela-Cardenas et al., J ImmunoTherapy of Cancer, 2022. 10(5): e004770; FIG. 7A) whose replication is dependent on the bioavailability of arginine. ASS1^{KO} tumors treated with MyxPD1/IL12 displayed a less pronounced response to viral therapy including earlier tumor relapse and poorer overall survival (FIG. 7B, FIG. 7C). Taken together, these data suggest that ASS1 status might affect the responsiveness of tumors to MYXV-based oncolytic virotherapy by restricting intratumoral viral replication.

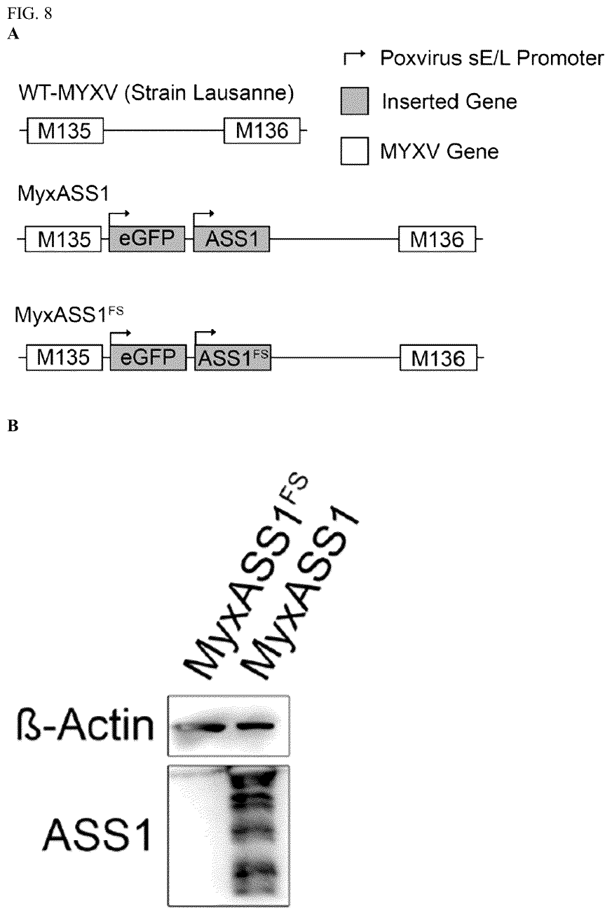

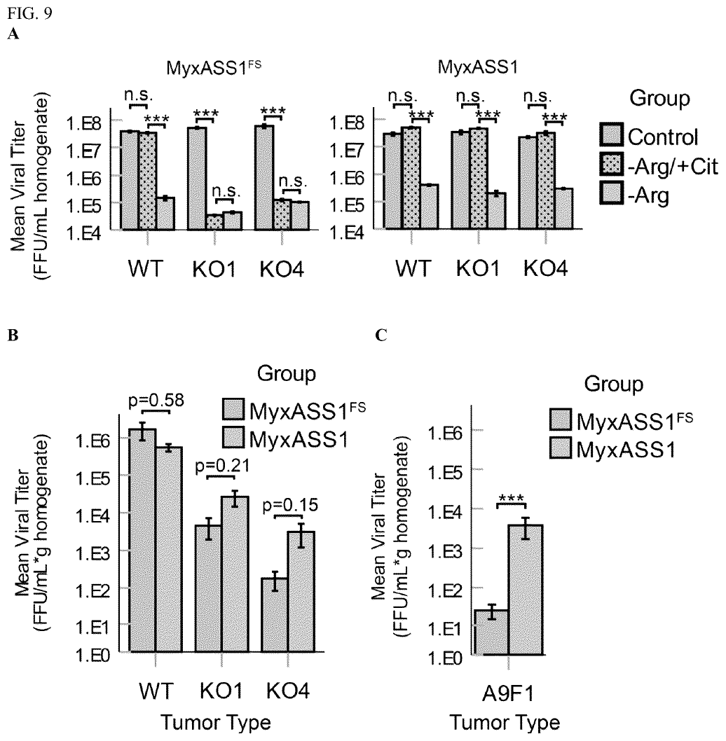

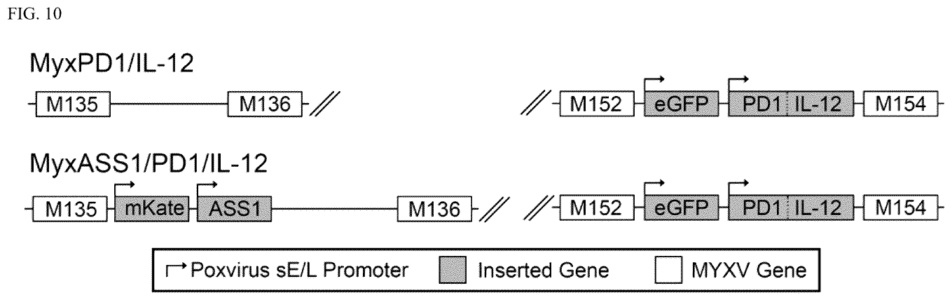

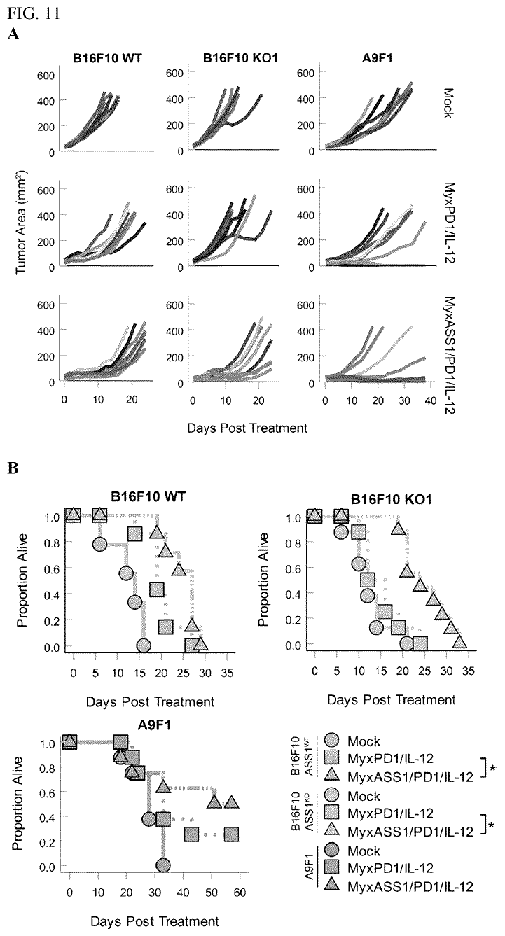

Viral reconstitution of arginine biosynthesis rescues both intratumoral replication and therapeutic efficacy. Since the tumor-intrinsic loss of ASS1 poses a barrier to both oncolytic virotherapy replication and therapeutic performance, reconstituting the arginine biosynthetic pathway by expressing ASS1 from a recombinant MYXV might improve treatment of ASS1-deficient tumors. To address this, a recombinant MYXV construct was generated that expressed both GFP and the murine ASS1 ORF (MyxASS1) (FIG. 8a, FIG. 8B). A control virus expressing both GFP and a frameshifted portion of the murine ASS1 ORF (MyxASS1^{FS}) was also generated as a control. In vitro testing confirmed that the viral replication of both MyxASS1 and MyxASS1F'S was inhibited by the removal of arginine from the growth media. However, replication of MyxASS1 could now be rescued in ASS1^{KO} B16F10 cells by adding citrulline while replication of MyxASS1^{FS} could not (FIG. 9A). Similarly, while MyxASS1 and MyxASS1F$ displayed similar viral titers in tumors derived from ASS1⁺ B16F10 cells, MyxASS1 displayed a strong trend towards increased viral titers in tumors derived from two distinct ASS1^{KO} B16/F10 cell lines (FIG. 9B) as well as significantly increased titers in tumors derived from intrinsically ASS1-deficient A9F1 cells (FIG. 9C). Finally, to determine whether expression of ASS1 might improve MYXV-based OV, a triply-recombinant, therapeutically active virus expressing full-length ASS1 along with soluble PD1 and IL12 (MyxPD1/IL12/ASS1) was generated (FIG. 10). Consistent with expression of ASS1 improving oncolytic virotherapy performance in Arg-auxotrophic tumors, treatment of tumors derived from ASS1^{KO} B16/F10 cells with MyxAss1/PD1/IL12 resulted in improved tumor control compared to treatment with a non-ASS1 expressing control virus (FIG. 11A, FIG. 11B). Similar results were observed in mice bearing tumors derived from intrinsically ASS1-deficient A9F1 cells although in this model the improve therapeutic efficacy presented as a higher percentage of complete responders and not an increase in median survival time (FIG. 11A, FIG. 11B). Collectively, these results demonstrate that defects to oncolytic virus replication and performance induced by the loss of ASS1 expression from malignant tumor cells may be, at least partially, overcome by the reconstitution of this pathway using recombinant viruses.

The efficacy of many oncolytic virotherapies is influenced by the successful replication of the viral agent. However, despite this dependence, the barriers to this replication imposed by the tumor microenvironment remain incompletely understood. An intact antiviral response represents one major obstacle to achieving effective infection within treated tumors. However, even within the context of severe immunodeficiency, such as immune deficient NOD. Cg-Prkdc/scid/Il2rg^{tm1Wj1/Sz}J mice, many oncolytic infections—including MYXV—still fail to achieve complete tumor eradication. Thus, it is likely that barriers to viral infection exist beyond the context of antiviral immunity. Determining the identity of these barriers therefore represents an opportunity to improve our basic understanding of oncolytic virotherapy and enhance its therapeutic potential. The data reported herein suggest that metabolic deficiencies resulting from dysregulation of arginine biosynthesis within malignant cells may contribute to oncolytic virotherapy resistance.

The metabolomic evaluation of the changes induced in B16F10 tumors by viral treatment (FIG. 1) demonstrate clear changes to the arginine biosynthetic pathway upon MYXV infection. Similarly, the in vitro data clearly demonstrate that MYXV infection is wholly dependent on the presence of bioavailable arginine in multiple cell lines (FIG. 2). The mechanism(s) through which arginine deprivation inhibits the replication of potential oncolytic candidates (e.g., MYXV) remain unclear.

Regardless of the precise mechanism(s) responsible for inhibiting MYXV replication in arginine-limited conditions, ASS1 competency had a clear, robust effect on both viral replication and therapeutic efficacy in vivo (FIGS. 5-7). This suggests that bioavailable arginine is inherently limiting within the tumor microenvironment of both B16F10 and A9F1 tumors and that enough citrulline is present to allow for arginine biosynthesis in arginine-autotrophic cells. Even in ASS1^{KO} tumors, some evidence of MYXV replication was still observed (FIG. 7). This replication occurred in significantly fewer distinct viral foci, with the majority being much smaller and dimmer than their counterparts in ASS1^{WT} tumors. However, a few foci in ASS1^{KO} tumors were individually comparable in size and GFP intensity to those found in ASS1 WT tumors. This observation suggests that loss of ASS1 decreases the portion of a tumor that is amenable to infection. These results potentially indicate that arginine bioavailability could be heterogeneous throughout the tumor, and if so, recapitulate the observations in vitro that infection can proceed unabated in ASS1^{KO} cell lines so long as arginine is available exogenously.

While loss of ASS1 from tumor cells reduced the efficacy of MYXV-based oncolytic virotherapy, this effect could be partially rescued by arming recombinant viruses with ASS1 (FIGS. 8-11). It is unclear how the increased viral replication seen in ASS1-expressing MYXV-constructs results in improved tumor control. One model is that increased viral replication causes more direct viral lysis of infected tumors cells. This model, however, is difficult to rationalize with the observation that the total amount of MYXV infection within a tumor rarely exceeds ˜5-10% even in ASS1-competent settings. An alternative model is that the induction of the anti-tumor immune responses required to cause tumor regression in MYXV-based oncolytic virotherapy likely represents thresholded events. In this model, the increased replication observed for ASS1-expressing viruses allows them to cross a defined threshold which then induces a better therapeutic response. A third model is that the tumor control effect of ASS1-expressing MYXV constructs could be explained by more robust expression of a recombinant virus's therapeutic transgenes. For example, increased replication of MyxAss1/PD1/IL 12 could result in locally increased concentrations of IL 12 which render the treatment more effective. Finally, decreased consumption of bioavailable arginine resulting from viral reconstitution of arginine biosynthesis may also liberate arginine content for use by other cells, such as T cells, that depend on arginine for proper function.

Thus, the bioavailability of arginine affects the replication of numerous viruses used as oncolytic agents. The lack of arginine within the tumor microenvironment is a potential barrier to oncolytic virotherapy performance. As demonstrated herein, altered arginine metabolism within the tumor microenvironment represents a barrier that can inhibit the performance of oncolytic virotherapy. The present disclosure provides tools and methods to increase the availability of arginine within the tumor microenvironment to increase the efficacy of oncolytic virotherapy. In particular, the present disclosure provides viruses expressing ASS1 that allow infected cells to synthesize arginine. In addition, the present disclosure includes tools and methods to study the metabolic mechanisms that affect oncolytic virotherapy in tumors. In particular, the present disclosure includes tools and methods to study the effect of arginine biosynthesis and bioavailability on oncolytic virotherapy. As used herein, “biosynthesis” of arginine refers to enzymatic synthesis of arginine, often within a cell.

The present disclosure identifies metabolic pathways that affect the in vivo replication of oncolytic viruses by characterizing changes to metabolites in the tumor microenvironment following viral treatment. Thus, in one aspect, the present disclosure relates to a recombinant virus. The recombinant virus may be engineered to express one or more exogenous genes. The one or more exogenous genes may be associated with amino acid biosynthesis. In one or more certain embodiments, the one or more exogenous genes may promote and/or facilitate arginine biosynthesis.

As described herein, decreased arginine bioavailability is associated with decreased efficacy of viral replication. Many solid tumors have relatively low levels of bioavailable arginine and decreased expression of proteins associated with arginine biosynthesis. Thus, it may be desirable to increase arginine biosynthesis within the tumor to increase viral replication. This may result in higher efficacy of oncolytic virotherapy.

One possible method of increasing arginine biosynthesis within a tumor is to increase expression of proteins associated with arginine biosynthesis. Infecting a tumor cell with a virus including an expression cassette encoding a protein associated with arginine biosynthesis may increase expression of the protein within the tumor cell. Thus, in one or more embodiments, a recombinant virus of the present disclosure includes an expression cassette encoding a protein associated with arginine biosynthesis. For example, one exemplary protein associated with arginine biosynthesis is argininosuccinate synthetase 1 (ASS1).

The expression cassette includes an open reading frame (ORF). The ORF includes a coding region encoding a protein associated with arginine biosynthesis. Human coding regions associated with arginine biosynthesis include those shown in FIG. 1C, such as argininosuccinate lyase (ASL), argininosuccinate synthase 1 (ASS1), and ornithine transcarboxylase (OTC). In one or more certain embodiments, the ORF may include a homolog or ortholog of a human coding region associated with arginine biosynthesis, such as a murine homolog.

In one or more embodiments, the expression cassette includes genetic regulatory elements and the coding region encoding a protein associated with arginine biosynthesis. Genetic regulatory regulation elements may include, for example, a promoter, a post-translational regulatory element, a polyadenylation site, or an enhancer. The promoter may be, but is not limited to, a poxvirus early/late promoter, a herpes simplex virus IE4/5 promoter, a cytomegalovirus (CMV) promoter, a CAG promoter, or an SV40 promoter. The regulatory element may be, for example, woodchuck hepatitis virus posttranscriptional regulatory element (WPRE). In one or more embodiments, the virus may include one or more additional elements such as a fluorescent protein, an affinity tag, a localization tag, or an additional element for detection of expression of a coding region, such as fluorophore. Suitable fluorophores include, for example, GFP (e.g., eGFP, sfGFP), RFP, YFP, mCherry, and m Venus.

While exemplified herein in the context of an illustrative embodiment in which the recombinant virus is derived from a myxoma virus, the compositions and methods described herein can involve any suitable virus whose replication depends on arginine. Exemplary viruses from which the recombinant virus may be derived include, but are not limited to, a myxoma virus, a herpesvirus (e.g., talimogene laherparepvec), an orthopox virus (e.g., vaccinia virus), an adenovirus, a parvovirus, an alphavirus (e.g., Semliki Forest virus), a flavivirus (e.g., zika virus), a paramyxovirus (e.g., measles virus, Newcastle disease virus), a picornavirus (e.g., Seneca Valley virus), a poliovirus, a reovirus, a rhabdovirus (e.g., vesicular stomatitis virus), or a filovirus (e.g., Marburg virus). The virus is generally replication competent, meaning that the virus is capable of replicating within an infected cell.

### Immune Checkpoint Inhibition and Oncolytic Virotherapy

In one or more embodiments, a recombinant virus of the present disclosure includes an expression cassette encoding an immune checkpoint inhibitor. An exemplary recombinant virus including an expression cassette encoding an immune checkpoint inhibitor is described in PCT Publication No. WO 2021/168186 A1, incorporated by reference in its entirety herein. This disclosure further describes compositions and methods in which a recombinant virus including an expression cassette encoding an immune checkpoint inhibitor is used in combination with a recombinant virus including an expression cassette encoding a protein associated with arginine biosynthesis for oncolytic virotherapy.

In one or more embodiments, a recombinant virus of the present disclosure includes an expression cassette encoding a form of programmed death protein 1 (PD1). In one or more embodiments, a recombinant virus of the present disclosure includes an expression cassette encoding interleukin 12 (IL-12). In one or more embodiments, a recombinant virus of the present disclosure includes an expression cassette encoding a tumor antigen or a cytokine/chemokine other than IL-12. In one or more embodiments, a recombinant virus includes a first expression cassette encoding a soluble form of PD1 and a second expression cassette encoding IL-12. Optionally, a recombinant virus includes an expression cassette encoding a tumor antigen or a cytokine/chemokine other than IL-12. In one or more certain embodiments where a recombinant virus includes more than one expression cassette, two of the expression cassettes are provided as a dicistronic expression cassette.

In one or more embodiments, the virus includes a cytokine/chemokine selected from the group consisting of IL-2, IL-4, IL-15, IL-17, IL-18 (mutated), IL-23, IL-35, IL-36, IFN-γ, IFN-β, RANTES/CCL5, GM-CSF, cGAS, or Ebola GP (aa1-298). In one or more certain embodiments, the virus includes a tumor antigen selected from the group consisting of p53, MUC1, PSMA, mRAS or S100P.

In one or more embodiments, the soluble PD1/mutant soluble PD1 includes an extracellular region of human PD1. In one or more embodiments, the soluble PD1/mutant soluble PD1 and the IL-12 are encoded in the dicistronic expression cassette. For example, the soluble PD1/mutant soluble PD1 and the IL-12 may be encoded in distinct expression cassettes. In one or more embodiments, the soluble PD1/mutant soluble PD1 and the chemokine/cytokine are encoded in the dicistronic expression cassette. In one or more embodiments, the soluble PD1/mutant soluble PD1 and the chemokine/cytokine are encoded in distinct expression cassettes. In one or more certain embodiments, the soluble PD1/mutant soluble PD1 and the tumor antigen are encoded in the dicistronic expression cassette. In one or more certain embodiments, the soluble PD1/mutant soluble PD1 and the tumor antigen are encoded in distinct expression cassettes. In one or more embodiments, the IL-12 and the tumor antigen are encoded in the dicistronic expression cassette. In one or more certain embodiments, the IL-12 and the tumor antigen are encoded in distinct expression cassettes.

In one or more embodiments, a recombinant virus of the present disclosure includes expression cassettes encoding ASS1, PD1, and IL-12. A dicistronic expression cassette may encode any two of ASS1, PD1, and IL-12. For example, a recombinant virus may include a first expression cassette encoding ASS1 and a second expression cassette encoding PD1 and IL-12, wherein the second expression cassette is a dicistronic expression cassette.

In another aspect, the present disclosure relates to a composition including more than one recombinant virus. A composition may include any recombinant virus described herein. A composition may include two, three, four, or five viruses. In one or more embodiments, a composition includes a first virus including an expression cassette encoding ASS1 and a second virus including an expression cassette encoding PD1 and IL-12.

### Methods of Treating a Cell

In another aspect, the present disclosure relates to a method of treating a cell, including contacting the cell with a recombinant virus. The recombinant virus includes an expression cassette encoding a protein associated with arginine biosynthesis. In one or more embodiments, the recombinant virus includes an expression cassette encoding ASS1. In one or more embodiments, the recombinant virus includes expression cassettes encoding ASS1, PD1, and IL-12. Any recombinant virus described herein may be used for this method.

In one or more embodiments, the cell is a cancerous cell. The cancerous cell may be part of a tumor, such as a solid tumor. The cancer may be, for example, melanoma, lung cancer, pancreatic cancer, ovarian cancer, or glioblastoma.

In one or more embodiments, the method includes contacting a cell with more than one type of recombinant virus. In one or more embodiments, the method includes contacting a cell with a first recombinant virus including an expression cassette encoding a protein associated with arginine biosynthesis and a second recombinant virus including an expression cassette encoding an immune checkpoint inhibitor. In one or more certain embodiments, the method includes contacting a cell with a first recombinant virus including an expression cassette encoding ASS1 and a second recombinant virus encoding PD1. In one or more certain embodiments, the method includes contacting a cell with a first recombinant virus including an expression cassette encoding ASS1 and a second recombinant virus including an expression cassette encoding IL-12. In one or more certain embodiments, the method includes contacting a cell with a first recombinant virus including an expression cassette encoding ASS1 and a second recombinant virus encoding a tumor antigen or a cytokine/chemokine other than IL-12.

In one or more embodiments, the method includes contacting a cell with a first recombinant virus including an expression cassette encoding ASS1 and a second recombinant virus including multiple expression cassettes encoding (a) a soluble form of programmed cell death protein 1 (PD1); (b) interleukin 12 (IL-12); and (c) a tumor antigen or a cytokine/chemokine other than interleukin 12 (IL-12).

### Methods of Predicting Clinical Efficacy of Oncolytic Virotherapy

In another aspect, the present disclosure describes a method of predicting the efficacy of an oncolytic virotherapy. Like many immunotherapies, oncolytic virotherapy often results in highly biphasic outcomes with some patients displaying impressive durable responses while others gain little to no benefit. Because of this bi-phasic nature, the ability to accurately predict patient outcomes can be useful to the successful application of oncolytic virotherapy.

In one or more embodiments, the present disclosure describes a method of interpreting ASS1 status to predict the success of oncolytic virotherapy in a patient. In one or more embodiments, the method includes obtaining a measurement of an ASS1 level in a patient and correlating the measurement with a likelihood of success of oncolytic virotherapy. In general, it is described herein that lower ASS1 levels are indicative of lower success rates of oncolytic virotherapy. The oncolytic virotherapy may include treatment with a virus consistent with those described herein, such as a replication competent myxoma virus.

A component is said to be present in amounts “no more than” a reference amount or concentration when the component is not absent but is present in an amount up to the reference amount or concentration.

As used herein, the term “nucleic acid” or “oligonucleotide” refers to polynucleotides such as deoxyribonucleic acid (DNA) or ribonucleic acid (RNA). Nucleic acids include but are not limited to genomic DNA, cDNA, RNA, iRNA, miRNA, tRNA, ncRNA, rRNA, and recombinantly produced and chemically synthesized molecules such as aptamers, plasmids, anti-sense DNA strands, shRNA, ribozymes, nucleic acids conjugates, and oligonucleotides. A nucleic acid may be single-stranded, double-stranded, linear, or covalently circularly closed molecule. A nucleic acid can be isolated. The term “isolated nucleic acid” means that the nucleic acid (i) was amplified in vitro, for example via polymerase chain reaction (PCR), (ii) was produced recombinantly by cloning, (iii) was purified, for example, by cleavage and separation by gel electrophoresis, (iv) was synthesized, for example, by chemical synthesis, or (vi) extracted from a sample. A nucleic might be introduced—i.e., transfected-into cells. When RNA is used to transfect cells, the RNA may be modified by stabilizing modifications, capping, or polyadenylation.

Generally, nucleic acid can be extracted, isolated, amplified, or analyzed by a variety of techniques such as those described by Green and Sambrook, Molecular Cloning: A Laboratory Manual (Fourth Edition), Cold Spring Harbor Laboratory Press, Woodbury, NY 2,028 pages (2012); or as described in U.S. Pat. Nos. 7,957,913; 7,776,616; 5,234,809; and 9,012,208. Examples of nucleic acid analysis include, but are not limited to, sequencing and DNA-protein interaction. Sequencing may be by any method known in the art. DNA sequencing techniques include classic dideoxy sequencing reactions (Sanger method) using labeled terminators or primers and gel separation in slab or capillary, and next generation sequencing methods such as sequencing by synthesis using reversibly terminated labeled nucleotides, pyrosequencing, 454 sequencing, Illumina/Solexa sequencing, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, polony sequencing, and SOLID sequencing. Separated molecules may be sequenced by sequential or single extension reactions using polymerases or ligases as well as by single or sequential differential hybridizations with libraries of probes.

The subject can be a human or a non-human animal such as, for example, a livestock animal, a laboratory animal, or a companion animal. Exemplary non-human animal subjects include, but are not limited to, animals that are hominid (including, for example chimpanzees, gorillas, or orangutans), bovine (including, for instance, cattle), caprine (including, for instance, goats), ovine (including, for instance, sheep), porcine (including, for instance, swine), equine (including, for instance, horses), members of the family Cervidae (including, for instance, deer, elk, moose, caribou, or reindeer), members of the family Bison (including, for instance, bison), feline (including, for example, domesticated cats, tigers, lions, etc.), canine (including, for example, domesticated dogs, wolves, etc.), avian (including, for example, turkeys, chickens, ducks, geese, etc.), a rodent (including, for example, mice, rats, etc.), a member of the family Leporidae (including, for example, rabbits or hares), members of the family Mustelidae (including, for example ferrets), or member of the order Chiroptera (including, for example, bats).

“Treat” or variations thereof refer to reducing, limiting progression, ameliorating, or resolving, to any extent, the symptoms or signs related to a condition. A “treatment” may be therapeutic. “Therapeutic” and variations thereof refer to a treatment that ameliorates one or more existing symptoms or clinical signs associated with a condition. Generally, a “therapeutic” treatment is initiated after the condition manifests in a subject.

Treating a condition can be initiated after the subject exhibits one or more symptoms or clinical signs of the condition. Treatment may also be continued after symptoms have resolved, for example to prevent or delay their recurrence. Accordingly, a composition can be administered at the time or after the time when the subject first exhibits a symptom or clinical sign of the condition (e.g., cancer). Treatment initiated after the subject first exhibits a symptom or clinical sign associated with the condition may result in decreasing the severity of symptoms and/or clinical signs of the condition compared to a subject to which the composition is not administered, and/or completely resolving the condition.

Thus, the method includes administering an effective amount of the composition to a subject having, or at risk of having, a particular condition, such as cancer. In this aspect, an “effective amount” is an amount effective to reduce, limit progression, ameliorate, or resolve, to any extent, a symptom or clinical sign related to the condition.

Reference throughout this specification to “one embodiment,” “an embodiment,” “certain embodiments,” “one or more embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, particular embodiments may be described in isolation for clarity. Thus, unless otherwise expressly specified that the features of a particular embodiment are incompatible with the features of another embodiment, the particular features, configurations, compositions, or characteristics may be combined in any suitable manner in one or more embodiments. Thus, features described in the context of one embodiment may be combined with features described in the context of a different embodiment except where the features are necessarily mutually exclusive.

The words “preferred” and “preferably” refer to embodiments of the invention that may afford certain benefits under certain circumstances. However, other embodiments may also be preferred under the same or other circumstances. Furthermore, the recitation of one or more preferred embodiments does not imply that other embodiments are not useful and is not intended to exclude other embodiments from the scope of the invention.

As used herein, the word “exemplary” means to serve as an illustrative example and should not be construed as preferred or advantageous over other embodiments.

### Examples

The present invention is illustrated by the following examples. It is to be understood that the particular examples, materials, amounts, and procedures are to be interpreted broadly in accordance with the scope and spirit of the invention as set forth herein.

### Cell Lines and Reagents

Both the BSC40 and B16F10 cells lines were purchased from the American Type Culture Collection (Manassas, VA). Lewis Lung Cancinoma-A9F1 cells (A9F1) were a generous gift from Dr. Mark Rubinstein at the Medical University of South Carolina. ASS1^{KO} B16F10 cell lines were generated using a CRISPR/Cas9 system targeting murine ASS1 (gRNA sequence: TCAGGCCAACATTGGCCAGA (SEQ ID NO:1); plasmid: PX459, GenScript Biotech, Piscataway, NJ) as previously described (Bartee et al., J Immunother Cancer, 2019. 7(1): 11; Bartee et al., Cancer Research, 2017. 77(11):2952). B16F10 cells treated with a scrambled gRNA (referred to in this paper as WT cells) have been previously described (Bartee et al., J Immunother Cancer, 2019. 7(1):11). Cell lines were maintained in complete Dulbecco's Modified Eagle Medium (DMEM) (Corning, Inc., Corning, NY) supplemented with 10% fetal bovine serum (FBS) and 1× penicillin/streptomycin/glutamine (Corning, Inc., Corning, NY). The FBS lot used in this study did not contain functionally relevant levels of residual Arg or Citrulline. Media lacking each amino acid was created by starting with DMEM lacking all essential amino acids (United States Biological, Swampscott, MA) and supplementing it with all the amino acids typically found in DMEM barring the amino acid of interest. Media specifically lacking Arg (−Arg) was created by starting with DMEM lacking Arg (United States Biological, Swampscott, MA) and supplementing it with 3.7 g/L sodium bicarbonate and the specified concentrations of filter-sterilized L-Arg-HCl (VWR International, LLC, Radnor, PA). Arg-free media reconstituted with 400 μM L-Arg-HCl was used for all control conditions. For metabolic rescue, Arg-free media was supplemented with either 400 μM L-Citrulline or AS (Sigma-Aldrich, St. Louis, MO). For all in vitro experiments involving-Arg groups and MYXV infection, cells were cultured in-Arg media 24 hours prior to infection. Cultures were checked quarterly for mycoplasma contamination using PCR. The following primary antibodies were used for Western blotting in this study: ASS1 (D404B, Cell Signaling Technology, Danvers MA) ornithine transcarbamylase (OTC) (NBP1-31582, Novus Biologicals, Centennial CO), argininosuccinate lyase (ASL) (NBP1-32752, Novus Biologicals, Centennial CO), β-actin (13E5, Cell Signaling Technology, Danvers MA). HRP-linked Anti-Rabbit IgG (7074S, Cell Signaling Technology, Danvers MA) was used as the secondary antibody for all blots.

### Viral Constructs, Infection, and Quantification of Virus

All constructs were based on the Lausanne strain of MYXV (Stanford et al., Molecular Therapy, 2008. 16(1): 52-59; Rahman, M. M. and G. McFadden, J Clin Med, 2020. 9(1)). Recombinant MYXV expressing green fluorescent protein (MyxGFP) has been previously described (Bartee et al., Cancer Research, 2017. 77(11):2952; Valenzuela-Cardenas et al., J Immuno Therapy of Cancer, 2022. 10(5): e004770). Constructs generated herein include MYXV encoding full-length murine ASS1 (MyxASS1), MYXV encoding a non-functional, frame shifted murine ASS1 (MyxASS1^{FS}), and MYXV encoding the soluble ectodomain of murine PD1, an IL 12 fusion protein, and full-length murine ASS1 (MyxPD1/IL12/ASS1).

To generate MyxASS1 and MyxASS1^{FS}, BSC40 cells were transfected with a pBLUESCRIPT plasmid (Agilent Technologies, Inc., Santa Clara, CA) encoding both GFP and the murine ASS1 open-reading-frame (ORF) located between fragments homologous to the MYXV m135r and m136r genes.

MyxASS1 was generated using the intact murine ASS1 ORF while MyxASS1F$ was generated using an ASS1 ORF lacking the first 85 base-pairs (which deletes the start codon and also introduces a frame shift). Cells were then infected with MYXV-Lausanne and clonal, recombinant viruses expressing GFP isolated as previously described (Valenzuela-Cardenas et al., J Immuno Therapy of Cancer, 2022. 10(5):e004770).

MyxPD1/IL12/ASS1 was generated through a two-step process. In the first step, BSC40 cells were transfected with a pBLUESCRIPT plasmid (Agilent Technologies, Inc., Santa Clara, CA) encoding both GFP and a fusion protein encoding the soluble ectodomain of murine PD1 (aa 1-167) linked to a IL12 (p35-p40 fusion) by a P2A self-cleaving peptide linker located between fragments homologous to the MYXV m152r and m154r genes. Cells were then infected with MYXV-Lausanne and clonal recombinant viruses expressing GFP isolated as previously described (Valenzuela-Cardenas et al., J Immuno Therapy of Cancer, 2022. 10(5): e004770). In the second step, BSC40 cells were transfected with a pBLUESCRIPT plasmid (Agilent Technologies, Inc., Santa Clara, CA) encoding both mKATE and the murine ASS1 open-reading-frame (ORF) located between fragments homologous to the MYXV m135r and m136r genes. Cells were then infected with the previously generated MyxPD1/IL12 construct and clonal recombinant viruses expressing mKATE isolated as previously described (Valenzuela-Cardenas et al., J Immuno Therapy of Cancer, 2022. 10(5): e004770). Following clonal isolation, all constructs were amplified in BSC40 cells and purified using gradient centrifugation as previously described (Smallwood et al., Curr Protoc Microbiol, 2010. Chapter 14:Unit 14A 1).

For all in vitro infections, the desired amount of virus was added directly to cell cultures without media exchange so as not to disturb existing Arg content within the culture. To quantitate infectious virus (from all in vitro and in vivo samples as well as produced viral stock), cell pellets from either cell culture or infected tumor samples were freeze-thawed over three cycles in liquid nitrogen and a 37° C. water bath. Pellets were frozen a fourth time in liquid nitrogen and then thawed/sonicated for three minutes. Resulting homogenates were serially diluted and plated onto confluent BSC40s. The number of GFP foci was then quantified 48 hours post-infection and used to determine the titer of original samples.

### MTT Assay

Cells were plated at 10% confluency in a 96-well plate and left to incubate for 24 hours post-splitting. Media was then exchanged with either control, +citrulline/−Arg, +AS/−Arg, or −Arg media where applicable. At the indicated time points, MTT substrate (CellTiter 96 Non-Radioactive Cell Proliferation Assay, Promega, Madison, MI) was added at a 1:100 ratio to each well, and plates incubated for 1-1.5 hours at 37° C. 100 μL of stop solution was then added and the well contents homogenized via repeated pipetting. Total absorbance at 595 nm was then measured on a plate reader (SYNERGY NEO2, BioTek Instruments, Winooski, VT).

### Mouse Models

Female C57Bl/6J mice aged 6-10 weeks were obtained from The Jackson Laboratory (Bar Harbor, ME) and seeded subcutaneously (SQ) with 1×10⁶ tumor cells in 50 μL of cold phosphate buffered saline (PBS). Tumors were allowed to establish until they reached ˜25 mm² prior to use. Mice that did not establish consistent tumors were removed before the initiation of the experiment. In experiments evaluating viral replication and infection burden, mice were treated with a singular dose of 1×10⁶ foci forming units (FFU) of the indicated MYXV construct injected intratumorally in 50 μL of PBS. In experiments measuring therapeutic response, mice were treated with three doses of 1×10⁵ FFU of the indicated MYXV construct injected intratumorally in 50 μL of PBS. This dose was selected as previous experiments indicated that it yields tumor control but infrequent cures making it an ideal starting point to compare gain or loss of therapeutic response. Therapeutic doses were administered every other day over five days (QOD). Mice were euthanized when their tumor area exceeded 400 mm² (determined by measuring length×width with digital calipers). For all in vivo experiments, each data point represents a singular sample taken from an individual mouse bearing a single tumor. All experiments were approved by the University of New Mexico Health Science Center institutional animal care and use committee under protocol #20-201002-HSC.

### Histology

Tumors harvested from mice at the specified times were sagittally bisected with a scalpel, embedded in optical coherence tomography media, and frozen in liquid nitrogen-chilled isopentane for cryo-sectioning. Sections of tumors were cut on a cryostat at 8 μm section thickness. One section per tumor was taken for quantification. Images were collected on an Evos M5000 microscope using the GFP filter cube. Final images were used to quantify infection area, foci count, and foci characteristics in Fiji (Schindelin et al., Nature Methods, 2012. 9(7):676-682).

### Metabolomics

For metabolic analysis of virally treated tumors, B16F10 tumors were established on syngeneic C57Bl/6J mice for seven days. Tumors were then treated with a single dose of 1×10⁶ FFU of MyxGFP delivered directly intratumorally. Tumors were harvested four days post viral treatment (day 11 post initial implantation) and processed for analysis. For metabolic analysis of ASS1^{WT} and KO tumors, B16F10 tumors were established on syngeneic C57Bl/6J mice. Tumors were harvested 11 days after implantation and processed for analysis. For processing, tumors were immediately snap-frozen in liquid nitrogen at point of harvest. Frozen tumors were weighed, and then broken into coarse homogenates using mechanical percussion in liquid nitrogen cooled vessels. 30-50 mg of frozen coarse homogenates were then suspended in 80% methanol at 20 μL/mg tissue. Suspensions were sonicated, centrifuged at 12,000×g, and the resulting supernatants analyzed via LC-MS by the Metabolomics Core Facility at Robert H. Lurie Comprehensive Cancer Center of Northwestern University (Chicago, IL) as previously described (Weinberg et al., Nature, 2019. 565(7740):495-499). Metabolites without separable peaks or with unconfirmed identities were pruned prior to analysis (304 species down to 226 species), samples were normalized against total-ion current and comparisons drawn between groups across peak area. Analysis of metabolomics data was performed using metaboanalyst (Xia et al., Nucleic Acids Research, 2009. 37(suppl_2):W652-W660) on log₁₀-transformed data sets with auto-scaling (mean centered; divided by standard deviation of each variable). Global Test and relative-betweenness centrality were selected for enrichment method and topology analysis, respectively.

### Statistical and Informatic Methods

Principal component analyses (PCAs) were performed in R (v4.0.2) using the ggbiplot package for visualization. All other statistical analyses were performed in the statistical package for the social sciences (SPSS) (IBM, Armonk, NY). For statistical tests comparing two groups, unpaired Student's t-tests were used to determine significance (α=0.05). For statistical tests comparing more than two groups, ANOVAs with Tukey's HSD (Levene p<0.05) or Games-Howell (Levene p<0.05) post-hoc tests were used to determine significance (α=0.05). Differences in animal survival were determined using Log-Rank analysis (p<0.05). The specific test used for each analysis is notated within the figure legend. All error bars indicate the standard error of the mean (SEM) unless noted otherwise in figure legends.

### Metabolic Characterization of Tumors

A recombinant myxoma virus expressing GFP (vGFP) was prepared and titer was measured in foci-forming units (FFU). B16/F10 tumors were established in syngeneic mice and then treated with either saline or a single intratumoral (IT) bolus of 1×10⁷ FFU of vGFP. Four days post-treatment, the abundance of multiple metabolites including arginine within the treated tumors was determined using tandem mass-spectrometry. The abundance of arginine in muscle and kidney of each mouse was additionally measured.

Pathway analysis of the resulting dataset revealed that the most significantly altered metabolic pathways following vGFP treatment were related to arginine and proline metabolism (FIG. 1A). Tumors treated with vGFP had reduced levels of the urea cycle precursor L-citrulline as well as increased abundance of the downstream products ornithine, putrescine, and spermine (FIG. 1C).

The level of arginine in established B16/F10 tumors was measured and compared to that of muscle and kidney. Muscle and kidney each contained approximately 70 μM to 85 μM of arginine. In contrast, B16/F10 tumors contained less than 20 μM of arginine (FIG. 3B). From this example, it was learned that implanted tumors contained altered arginine and proline metabolism, and that tumors contained significantly lower levels of arginine than healthy tissue.

### Myxoma Virus Replication Requires Exogenous Arginine in Cultured Cells

BSC40 cells were grown in media lacking arginine or including increasing amounts of arginine (0 μM to 10 mM). Cells were infected with myxoma virus encoding GFP (vGFP). the FFU of cells in each condition was measured over 50 hours after infection to track viral replication. (FIG. 2B). Cells grown in media without arginine demonstrated significantly lower levels of myxoma virus infection as demonstrated by a loss of GFP expression (FIG. 2A). This suggests that myxoma virus replication requires bioavailable arginine, and that arginine within a cell is not sufficient to support myxoma virus replication.

In addition, myxoma virus did not replicate well in cells grown in media containing less than 100 μM of arginine. (FIG. 2C, FIG. 3A). Taken together, these data suggest that the limited bioavailability of arginine within solid tumors, particularly in solid tumors that contain less than 100 μM of arginine, may decrease oncolytic myxoma virus replication.

### Cells Lacking Arginine Biosynthetic Capacity Due to Loss of ASS1 Fail to Support Replication of Oncolytic Myxoma Virus

CRISPR/Cas9 was used to generate a series of functionally ASS1^{-/-} versions of the normally ASS1⁺ B16/F10 cell line. These cells were phenotypically similar to WT B16/F10s (FIG. 4A) but expressed a truncated form of ASS1 (FIG. 4B).

Both ASS1^{WT} and ASS1^{-/-} cell lines grown in media lacking arginine did not divide and were not able to support myxoma virus replication. However, when ASS1^{WT} cells were grown in media supplemented with citrulline, a metabolic precursor of arginine, the cells were able to divide and support myxoma virus replication. When ASS1^{WT} cells were grown in media supplemented with citrulline, the cells were still not able to divide or support myxoma virus replication (FIG. 4B). However, when ASS1^{WT} cells were grown in media supplemented with argininosuccinate, a metabolite derived from arginine, the cells were able to divide and support myxoma virus replication (FIG. 4B). This finding supports the idea that synthesis of arginine by ASS1 is required for efficient replication of myxoma virus in B16/F10 cells.

### Tumors Display Reduced Responsiveness to Myxoma Virus-Based Oncolytic Virotherapy

Mice were treated to implant ASS1^{-/-} or ASS1^{WT} B16/F10 tumors. Mice having each tumor were infected with vGFP or a myxoma virus construct expressing IL-12 and a PD1 inhibitor (vPD1/IL-12) (FIG. 10, FIG. 11). Creation of the vPD1/IL-12 virus is described in greater detail in PCT Publication No. WO 2021/168186. Six days post-treatment with oncolytic myxoma virus, ASS1^{-/-} tumors contained approximately two orders of magnitude less of replication competent virus than comparable ASS1^{WT} tumors (FIG. 8, FIG. 9) and also displayed a significantly poorer therapeutic response to treatment with vPD1/IL-12 (FIG. 10, FIG. 11).

Generation of vASS1 and Infection of ASS1^{-/-} or ASS1^{WT} Tumors with vASS1

It was investigated whether treatment with a myxoma virus encoding ASS1 would alter viral replication within a tumor. vASS1 was generated by recombining the murine ASS1 ORF into the unmodified myxoma virus (strain Lausanne) genome between the M135 and M136 viral open reading frames (FIG. 8). Following isolation and amplification, preliminary experiments with this construct demonstrated that it expressed the ASS1 protein (FIG. 9) and replicated in ASS1″ cells in the presence of citrulline (FIG. 9).

Treatment of Tumors with vPD1/IL-12 in Combination with Additional Viruses

To determine whether this deficiency could be remedied by expression of ASS1 by the myxoma virus, a recombinant myxoma virus expressing PD1, IL-12, and ASS1 (vPD1/IL-12/ASS1) was prepared. To prepare vPD1/IL-12/ASS1, the poxviral recombination plasmid used to generate vASS1, which is designed to incorporate both ASS1 and GFP into the intergenic region between the M135 and M136 viral ORFs, was homologously recombined with an existing bicistronic version of vPD1/IL-12 in which both PD1 and IL-12 (along with a TdTR marker gene) are encoded into the viral M153 loci. Recombinant vPD1/IL-12/ASS1 virus was isolated and purified. Purification, the replication properties, and cytopathic induction of vPD1/IL-12/ASS1 were validated in vitro.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “approximately” or “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

What is claimed:

1. A recombinant virus comprising an expression cassette encoding a protein associated with arginine biosynthesis, wherein replication of the recombinant virus is dependent on arginine.

2. The recombinant virus of claim 1, wherein the protein associated with arginine biosynthesis comprises argininosuccinate synthase 1 (ASS1), argininosuccinate lyase (ASL), or ornithine transcarboxylase (OTC).

3. The recombinant virus of claim 1, wherein the recombinant virus is derived from an oncolytic virus.

4. The recombinant virus of claim 1, wherein the recombinant virus is derived from a myxoma virus.

5. The recombinant virus of claim 1, wherein the recombinant virus is derived from a herpesvirus.

6. The recombinant virus of claim 1, wherein the recombinant virus comprises at least two expression cassettes, wherein, cumulatively, the at least two expression cassettes encode:
argininosuccinate synthase 1 (ASS1), argininosuccinate lyase (ASL), or ornithine transcarboxylase (OTC);
a soluble form of programmed cell death protein 1 (PD1); and
interleukin 12 (IL-12);
wherein two of the expression cassettes are provided as a dicistronic expression cassette.

7. The recombinant virus of claim 6, wherein:
a first expression cassette encodes argininosuccinate synthase 1 (ASS1), argininosuccinate lyase (ASL), or ornithine transcarboxylase (OTC); and
a second expression cassette encodes PD1 and IL-12.

8. A composition comprising:
a first recombinant virus comprising an expression cassette encoding ASS1, argininosuccinate lyase (ASL), or ornithine transcarboxylase (OTC); and
a second recombinant virus comprising an expression cassette encoding PD1 and IL-12, wherein each of the first recombinant virus and second recombinant virus is replication competent.

9. The composition of claim 8, wherein at least one of the first recombinant virus and the second recombinant virus is derived from myxoma virus.

10. A method of treating a cell, the method comprising contacting a cell with the recombinant virus of claim 1.

11. A method of treating a cell, the method comprising contacting a cell with the composition of claim 8.

12. A method of increasing replication of an oncolytic virus in a tumor microenvironment, the method comprising introducing to the tumor microenvironment the recombinant virus of claim 1.

13. A method of increasing replication of an oncolytic virus in a tumor microenvironment, the method comprising introducing into the oncolytic virus an expression cassette encoding a protein associated with arginine biosynthesis, wherein the oncolytic virus is replication competent.

14. The method of claim 13, wherein the protein associated with arginine biosynthesis comprises argininosuccinate synthase 1 (ASS1), argininosuccinate lyase (ASL), or ornithine transcarboxylase (OTC).


SEQUENCE LISTING (1 sequence)

SEQ ID NO: 1 - RNA, 20 nt, synthetic construct
  source 1..20: mol_type = other RNA
tcaggccaacattggccaga